(19) United States
(12) Patent Application Publication
(10) Pub. No.: US 2010/0006451 A1
(43) Pub. Date: Jan. 14, 2010

(54) Biosensing device and method for detecting target biomolecules in a solution

(76) Inventors: Neil Gordon, Hampstead (CA); Cezar Morun, Kitchener (CA); Garry Palmateer, London (CA)

Correspondence Address: JACOBSON HOLMAN PLLC, 400 SEVENTH STREET N.W., SUITE 600, WASHINGTON, DC, 20004, US

(21) Appl. No.: 12/216,914

(22) Filed: Jul. 11, 2008

(51) Int. Cl. G01N 33/487; B28B 11/08

(52) U.S. Cl. 205/777.5; 204/403.01; 204/293; 264/293

(57) ABSTRACT

A biosensing device for detecting a presence of target biomolecules is provided, including at least one working electrode having a systematic array of nano-electrode wires projecting vertically from an electrode pad. The nano-electrode wires all have a same shape and size and are distributed non-randomly over the electrode pad. Biosensor probes are attached to the nano-electrode wire, each including a bioreceptor selected to bind with a complementary target biomolecule to create a binding event, and an electrochemical transducer transducing this binding event into an electrical signal conducted by the corresponding nano-electrode wire. A biosensing method using such a device is provided, as well as a fabrication method thereof.

### FIELD OF THE INVENTION

The present invention relates to the filed of biosensing more particularly concerns a biosensing device, a working electrode for such a device, a fabrication method and a method for detecting target biomolecules using such as biosensing device.

### BACKGROUND

Biological hazards are caused by minute life forms called microorganisms and include certain types of infective bacteria, viruses, protozoa, and substances derived from microorganisms, that invade and grow within other living organisms and cause disease. As the consumption of a small amount of pathogens can sicken or kill a living organism, biohazards are placing an enormous toll on humans, animals, the food chain, and the environment. The most effective way to prevent the spread of biohazards is to frequently test for the presence of pathogens in people, animals, insects, surfaces, water, air and the food chain, and then rapidly contain biohazards before transmission occurs. As there is no universal indicator for biohazards, each specific type of pathogenic bacteria, virus, protozoa and other species needs to be tested separately to determine its presence. This has created a demand for specific biotesting.

Detecting and identifying biological materials typically requires a capital-intensive laboratory, specialized equipment, costly materials, and labor-intensive processing. Biotesting can take several days or weeks as many steps are required including the collection and transportation of samples from remote locations. This is a problem since pathogens and infectious diseases can spread before the test results are known. As well, the high cost per test limits the number of tests that can be undertaken by government agencies, commercial organizations, and consumers due to budget constraints.

In addition to biotesting laboratories, there is a rapidly growing market focused on the identification of biological materials using biosensors, which are measuring devices that convert a biological interaction into a measurable electrical signal. Biosensors can operate independently of laboratories and be used in portable devices and wireless sensor networks. The lower infrastructure cost, reduced consumption of materials, and ease of use of biosensors can greatly reduce the cost per test when compared to laboratory testing and subsequently be in great demand in the future.

A biosensor typically includes a bioreceptor which is a known biological molecule that generates a response when it interacts with a corresponding target biological molecule, and a transducer which measures a change in property resulting from this interaction and then converts the change into an electrical signal. Biosensors are typically classified by the type of property change they are based on and the corresponding transducer technology. These include changes in: temperature (calorimetric biosensor), light output or absorbance (optical biosensor), mass (piezo-electric biosensor), size, shape and conductivity of a conductive channel in a field effect transistor (field effect biosensor), and electrical current or signal from the movement of electrons in a redox reaction or electrical potential from the release or absorption of ions (electrochemical biosensor). Among the biggest problems associated with current biosensors is an unacceptably poor accuracy due to a high percentage of false positive and/or false negative results compared with biotesting laboratories.

To address this shortcoming, there has been a recent use of nanotechnology, including carbon nanotubes to improve biosensor transducers. Nanotechnology by virtue of its small size, vastly improved functional properties, ability for parallel processing, and exceptional capabilities for converging with biotechnology and microtechnology, offers new materials, structures, and methods for improving the performance of biosensor transducers. Some approaches make use of carbon nanotubes in piezo-electric biosensors (U.S. patent application published under no 2003/0218224 (SCHLAF)) and field effect biosensors (published U.S. patent applications nos 2004/0058153 (REN), 2004/0132070 (STAR) and 2005/0184294 (ZHANG) and U.S. Pat. No. 6,905,655 (GABRIEL)) with varying degree of accuracy and suitability for specific applications.

There however remains a need for nanotechnology-based biosensor devices that deliver a lower percentage of incorrect results than the standard biosensors available, along with other operational benefits such as portability, multiplexing, ease of use, and lower cost per test.

### SUMMARY OF THE INVENTION

In accordance with a first aspect of the present invention, there is provided a working electrode on a bottom assembly of an electrochemical biosensing device. The working electrode includes an electrode pad defining an area of the working electrode, as well as a systematic array of nano-electrode wires projecting vertically from this electrode pad. The nano-electrode wires all have a same shape and size and are distributed non-randomly over the electrode pad. The working electrode further includes a plurality of biosensor probes each attached to an extremity of one of the nano-electrode wires opposite the electrode pad. Each biosensor probe includes a bioreceptor selected to bind with a complementary target biomolecule to create a binding event, and an electrochemical transducer transducing this binding event into an electrical signal conducted by the corresponding nano-electrode wire. The working electrode additionally includes an insulating layer extending over the electrode pad so as to surround the nano-electrode wires while exposing the biosensor probes.

A method for the fabrication, on a substrate, of a working electrode for a biosensing device is also provided. The method includes the following:
a) providing an electrode pad on a portion of the substrate, thereby defining an area of the working electrode;
b) providing a systematic array of nano-electrode wires projecting vertically from the electrode pad. The nano-electrode wires all have a same shape and size and are distributed non-randomly over the electrode pad;
c) depositing an insulating layer over the electrode pad and surrounding the nano-electrode wires;
d) processing a top surface of the working electrode to prepare top extremities of the nano-electrode wires to receive biosensor probes; and
e) attaching a plurality of biosensor probes to the top extremities of the nano-electrode wires. Each biosensor probe includes a bioreceptor, selected to bind with a complementary target biomolecule to create a binding event, and an electrochemical transducer transducing the binding event into an electrical signal conducted by the corresponding nano-electrode wire.

In accordance with another aspect of the invention, there is also provided an electrochemical biosensing device for detecting a presence of target biomolecules in a solution.

The biosensing device first includes a bottom assembly having at least one negative control electrode for measuring background noise in the solution, and at least one working electrode. Each working electrode includes an electrode pad defining an area of the working electrode, and a systematic array of nano-electrode wires projecting vertically from the electrode pad. The nano-electrode wires all have a same shape and size and are distributed non-randomly over the electrode pad a plurality of biosensor probes are further provided and each attached to an extremity of one of the nano-electrode wires opposite the electrode pad. Each biosensor probe includes a bioreceptor selected to bind with one of the target biomolecules to create a binding event, and an electrochemical transducer transducing the binding event into an electrical signal conducted by the corresponding nano-electrode wire. An insulating layer extends over the electrode pad so as to surround the nano-electrode wires while exposing the biosensor probes.

The biosensing device further includes a top assembly extending over the bottom assembly and including a reference electrode and at least one counter electrode. A watertight compartment houses the top and bottom assemblies.

Measurement electronics are further provided for applying a scan of different potentials between electrodes in the top and bottom assemblies, and measuring electrical signals from each working electrode and each negative control electrode. Finally, the biosensing device includes related electronics for processing these electrical signals to determine therefrom the presence of the target biomolecules.

There is further provided a method for the fabrication of a bottom assembly for an electrochemical biosensing device for detecting a presence of target biomolecules in a solution, the method including the following:
a) providing a plurality of electrode pads on a substrate. The electrode pads define at least one negative control electrode for measuring background noise in the solution, and at least one working electrode;
b) providing a systematic array of nano-electrode wires on each of the electrode pads, the nano-electrode wires projecting vertically from the corresponding electrode pad. The nano-electrode wires of each of the systematic arrays all have a same shape and size and are distributed non-randomly over the corresponding electrode pad;
c) depositing an insulating layer over each of the electrode pad, surrounding the nano-electrode wires thereon;

for each of the working electrodes:
d) processing a top surface of the working electrode to prepare top extremities of the nano-electrode wires to receive biosensor probes; and
e) attaching a plurality of biosensor probes to the top extremities of the nano-electrode wires. Each biosensor probe has a bioreceptor selected to bind with a complementary target biomolecule to create a binding event, and an electrochemical transducer transducing this binding event into an electrical signal conducted by the corresponding nano-electrode wire.

In accordance with another aspect of the invention, there is provided a method for the fabrication of an electrochemical biosensing device for detecting a presence of target biomolecules in a solution. The method includes:
a) fabricating a bottom assembly according to the method stated above;
b) fabricating a top assembly, including providing a plurality of electrode pads on a substrate, the electrode pads defining at least one counter electrode, and one reference electrode;
c) joining the bottom assembly and top assembly within a water-proof housing;
d) connecting the electrodes of the bottom and top assemblies to measurement electronics for applying a scan of different potentials between electrodes in the top and bottom assemblies and measuring electrical signals from each working electrode and each negative control electrode; and
e) connecting the measurement electronics to related electronics for processing the electrical signals to determine therefrom the presence of the target biomolecules.

In accordance with yet another aspect of the invention, there is also provided a method for detecting a presence of target biomolecules in a solution using the biosensing device of defined above. The method includes:
a) exposing the bottom and top assemblies to the solution;
b) performing a first, a second and a third detection scan, whereby the potential difference between the electrodes of the top and bottom assembly is varied within a predetermined range in a same manner for each of these detection scans;
c) measuring a change of electrical signal from each of the working electrodes for the first and second detection scans;
d) measuring a change of electrical signal from each of the negative control electrodes for the second and third detection scans;
e) determining the presence of the target biomolecules from the changes of electrical signal measured at c) and d).

Other features and advantages of the present invention will be better understood upon reading of preferred embodiments thereof with reference to the appended drawings.

### BRIEF DESCRIPTION OF THE DRAWINGS

FIG. 1 is a schematic cross-sectional view of a working electrode according to an embodiment of the invention.

FIG. 2 is a top view of the working electrode of FIG. 1.

FIG. 3 is a schematic cross-sectional view of the top portion of a nano-electrode wire.

FIG. 4 a schematic cross-sectional view of the top portion of a nano-electrode wire showing a target biomolecule binding with a biosensor probe and generating a corresponding electrical signal.

FIGS. 5 and 6 are cross-sectional views of biosensing devices according to embodiments of the invention, respectively including a single and multiple counter electrodes, and negative control electrodes without and with biomolecular probes.

FIG. 7 is a flowchart illustrating a method for detecting the presence of target biomolecules according to an embodiment of the invention.

FIGS. 8a, 8b and 8c are graphs of the outcome of AC voltammetry scans from a biosensing device according to an embodiment of the invention showing the net signals at different potentials from a working electrode (FIG. 8a), from a negative control electrode (FIG. 8b), and the superimposed results from the two graphs illustrating the signal peak and the detection threshold (FIG. 8c).

FIG. 9 is a graph for converting the generated current peak into a concentration value.

FIG. 10 is a graph for converting the ratio of generated current peaks into a % viable value.

FIG. 11A is a flow chart illustrating a method for the fabrication of a biosensing device according to an embodiment of the invention; FIG. 11B is a flow chart illustrating the fabrication of the bottom assembly of such a device.

FIG. 12 is a photograph of a bottom assembly with 9 micro-electrode pads in a 3×3 array and micro-circuitry.

FIG. 13 is a photograph of a working electrode provided with a systematic array of nano-electrode wires.

### DESCRIPTION OF PREFERRED EMBODIMENTS OF THE INVENTION

Embodiments of the present invention will be described herein below in conjunction with the appended drawings, wherein like reference numerals refer to like elements throughout.

The embodiments of the present invention described herein relate to the detection of target biomolecules in a solution. The target biomolecules may be any analyte which one may wish to detect and which is apt to bind with a bioreceptor as described further below. The present invention may be particularly useful in the context of pathogen or biohazard detection such as specific strains of bacterium (e.g. E. coli, Salmonella, Vibrio cholerae), viruses (e.g. Hepatitis A, Norovirus), and protozoa (e.g. Cryptosporidium, Giardia). It is of course understood that the list above is given by way of example only and is in no way limitative to the scope of the present invention. In some embodiments of the present invention, a single biosensing device may be use to detect more than one type of target biomolecule. In addition to determining if target biomolecules are present, a given biosensing device may be used to evaluate the concentration of these biomolecules in the solution, as well as the percentage of the cells in the biomolecules that are viable and therefore capable of dividing and increasing in number. These and other optional features of the invention will be described further below with reference to examples of embodiments of the invention.

The solution containing the target molecules may be any fluid including an analyte solution from liquids, liquefied solids, or liquefied materials from air or gases which may include the target biomolecules. The solution may be pre-treated to increase the concentration of target biomolecules and to reduce the concentration of non-target biomolecules and undesired chemical molecules. Pre-treatment can include one or more of filtration, chemicals, electrochemical processes, lyses, heating, and sonication to produce a concentrated solution of target single strand nucleic acids or other biological materials that will interact with complementary biosensor probes of a biosensing device according to embodiments of the invention.

The biosensing devices described herein are preferably based on the same principles as electrochemical sensors as commonly used for the detection of chemicals. The expression “electrochemical sensor” refers to an electrochemical system that determines the presence and concentration of a chemical material through measurements of electrical signal in a solution between a working electrode and counter electrode such as induced by a redox reaction or electrical potential from the release or absorption of ions. The redox reaction refers to the loss of electrons (oxidation) or gain of electrons (reduction) that a material undergoes during electrical stimulation such as applying a potential. Redox reactions take place at the working electrode, also referred to as measuring electrode, which for chemical detection is typically constructed from an inert material such as platinum or carbon. The potential of the working electrode is measured against a reference electrode which is typically a stable, well-behaved electrochemical half-cell such as silver/silver chloride. The electrochemical system can be used to support many different techniques for determining the presence and concentration of the target biomolecules including, but not limited to, various types of voltammetry, amperometry, potentiometry and conductimetry such as AC voltammetry, differential pulse voltammetry, square wave voltammetry, electrochemical impedance spectroscopy, cyclic voltammetry, and fast scan cyclic voltammetry.

In order make an electrochemical biosensor, a working electrode adapted for the detection of biological material must be used. The section below describes a working electrode on a bottom assembly of a biosensing device according to an embodiment of the present invention.

Working Electrode

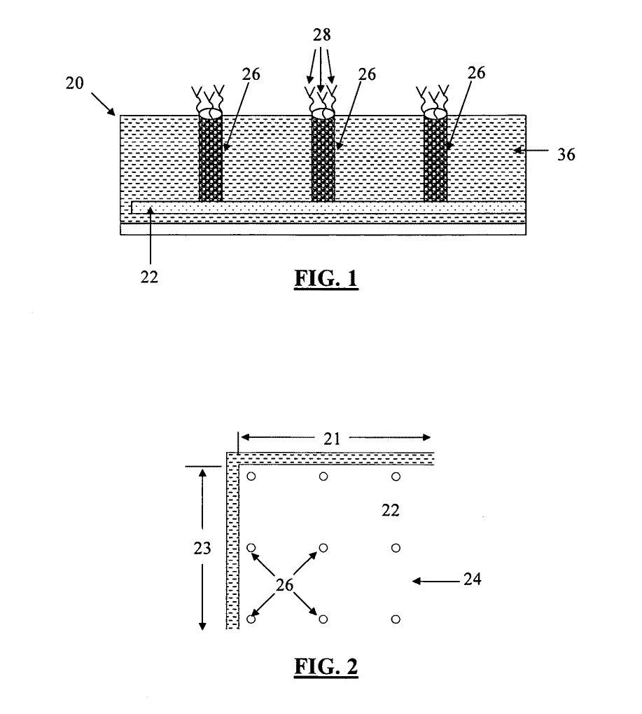

Referring to FIGS. 1 and 2 there is shown a working electrode 20 having an area defined by an electrode pad 22. The electrode pad 22 is made of a conductive or semi-conductive material such as chromium, platinum, titanium or related conductive metals, and preferably has dimensions on the micrometer scale. For example, the length 21 and width 23 of the electrode pad 22 may each be between about 0.5 and 2000 μm. In the illustrated embodiments the electrode pad 22 is shown to be square shaped, but other shapes such as rectangular can be considered without departing from the scope of the invention.

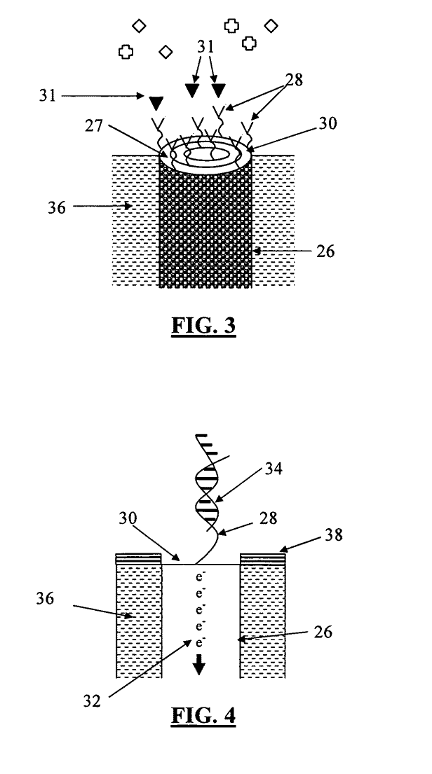

The electrode pad 22 is provided with a systematic array 24 of nano-electrode wires 26 projecting vertically therefrom. The nano-electrode wires can be embodied by carbon nanofibers having a structure defining several concentric walls 27 (see FIG. 3). Alternatively, they may be embodied by conductive or semi-conductive nanowires made of silicon, zinc oxide, tin indium oxide, copper, aluminum, indium, antimony or the like, or be shaped as any other structure such as nanotubes, nanocones, nanowhiskers or the like which can define a systematic array as mentioned above and is compatible with the other elements of the working electrode as defined below. The nano-electrode wires are preferably bamboo-like structured nanofibers, therefore having a have a circular radial cross-section, and all have a same diameter and height. The diameter of the nano-electrode wires is preferably between about 50 and 150 nm, and their height between about 0.1 and 20 μm, preferably 1 to 5 μm with a height to diameter aspect ratio between 20 and 60.

The expression “systematic array” is understood herein to refer to an arrangement of nano-electrode wires 26 all having a same shape and size and distributed non-randomly over the electrode pad 22, as opposed to a forest of nanostructured materials used as nano-electrode wires which are randomly positioned on an electrode pad and can have varying diameters, heights, shapes and distances between neighboring nano-electrode wires. It is understood that reference to a same shape and size for all of the nano-electrode wires can still encompass minor variations in these parameters from fabrication inasmuch as the operation of the working electrode 20 is not affected substantially.

In one embodiment of the invention, the nano-electrode wires 26 of the systematic array 24 have a same distance therebetween along two orthogonal axes, forming a squared array. This distance is preferably selected between about 1 μm and 5 μm. When inlaid disc-like nano-electrodes are produced from nanomaterials such as carbon nanotubes in “randomly distributed forests” as is known in the art, there is a hemispherical diffusion layer around each nano-electrode from close proximity or contact of neighboring nano-electrodes. This reduces the sensitivity and specificity of the nano-electrodes and can incorrectly generate false negative results. Moreover, the sensitivity of electrochemical biosensor transducers can be greatly improved when the diffusion layers of neighboring nano-electrodes are not overlapped so that each nano-electrode behaves as an independent nano-electrode and can conduct generated signals from its attached biosensor probes with minimal diffusion or noise from other nano-electrodes.

Li et al have demonstrated that nano-electrodes with spacing between neighboring nano-electrodes of at least 1 to 5 μm generate a sigmoidal shape in cyclic voltammetry measurements. By exploiting the minimum spacing requirement for diminished hemispherical diffusion, systematic arrays of nano-electrode wires can be successfully applied in cyclic voltammetry as well as other electrochemical biosensing techniques.

In one example of a working electrode according to an embodiment of the invention, nano-electrode wires all having about 80 nm in diameter and 3.2 μm in height extend upright and perpendicular to the electrode pad, with neighboring nano-electrode wires prearranged in a deliberate and non-random fashion with a spacing of 1 μm on the horizontal axis and 1 μm on the vertical axis. For such an example, a working electrode on an electrode pad of 200 μm×200 μm would have around 40,000 nano-electrode wires.

Referring to FIGS. 1, 3 and 4, each nano-electrode wire 26 is provided with one or more biosensor probes 28, attached to the top extremity 30 of the corresponding nano-electrode wire 26, that is, the extremity opposite the electrode pad 22. Each biosensor probe 28 includes a bioreceptor selected to bind with a complementary target biomolecule 31 to create a binding event, and an electrochemical transducer transducing this binding event into an electrical signal conducted by the corresponding nano-electrode wire 26. The biosensor probes are preferably 16S ribosomal RNA oligonucleotides or biomolecules. Alternatively the biosensor probes can for example be embodied by oligonucleotides, nucleic acids, peptides, ligands, proteins, enzymes, or any other material apt to bind with a complementary target biomolecule. In one embodiment, the same biosensor probe acts as both the bioreceptor and the transducer. In such a case, a binding event will generate electrons 32 through guanine oxidation of the biosensor probe 28 with a complementary target biomolecule, these electrons 32 being conducted by the corresponding nano-electrode wire 26 under an applied electric potential.

Referring more specifically to FIG. 4, an embodiment where the biosensor probe 28 is an oligonucleotide is schematically illustrated. The oligonucleotide probe 28 can bind and hybridize with a single strand 34 of a complementary nucleic acid from a target biomolecule in an analyte solution to form a double helix which facilitates the transduction of an electrochemical signal. About 10 to 1000 identical biosensor probes can for example fit on the tip of a single nano-electrode wire. In the example described above of a working electrode on a 200 μm×200 μm electrode pad having around 40,000 nano-electrode wires with a 1 micrometer pitch, a total of 400,000 to 40,000,000 biosensor probes for detecting a same target biomolecule can be provided. The use of multiple nano-electrode wires and biosensor probes has the benefit of redundancy and improved statistical reliability by averaging signals from many biosensors.

The working electrode 20 finally includes an insulating layer 36 which extends over the electrode pad 22 so as to surround the nano-electrode wires 26, while exposing the biosensor probes. The insulating layer 36 can for example be made of SiOₓ, Si_{y}N_{z}, epoxy, wax or parylene, where x, y and z are positive numbers.

With the working electrode described above, the bioreceptor and transducer functions only take place at the biosensor probes at the tips 30 of the nano-electrode wires 26. As the nano-electrode wires 26 merely function as electrical conductors that pass electrochemical signals to the electrode pad 22, the surface area surrounding the nano-electrode wires 26 can be completely insulated by the insulating layer 36 to improve specificity by preventing any contact by non-specific ions that cause false positive results. In the disclosed embodiment, the tips 30 of the nano-electrode wires 26 make up less than 1% of the surface area of the working electrode and the other 99% or more of the surface is constituted by the insulating layer 36, such as for example SiO₂. In some embodiments, the surface of the insulating layer 36 may be susceptible to non-specific adsorption of biomolecules that can produce false-positive results. To reduce the inaccuracy caused by non-specific adsorption, the insulation layer 36 may be passivated with protective moieties such as ethylene glycol, covering the insulating layer 36 with a thin passivation layer 38.

Biosensing Device

The working electrode described above may be used with any biosensing device apt to employ an electrochemical biosensing technique that exposes the working electrode to a solution containing target biomolecules, applies an electric potential to the working electrode, and measures the electrical signal generated by binding events of target biomolecules and complementary biosensor probes.

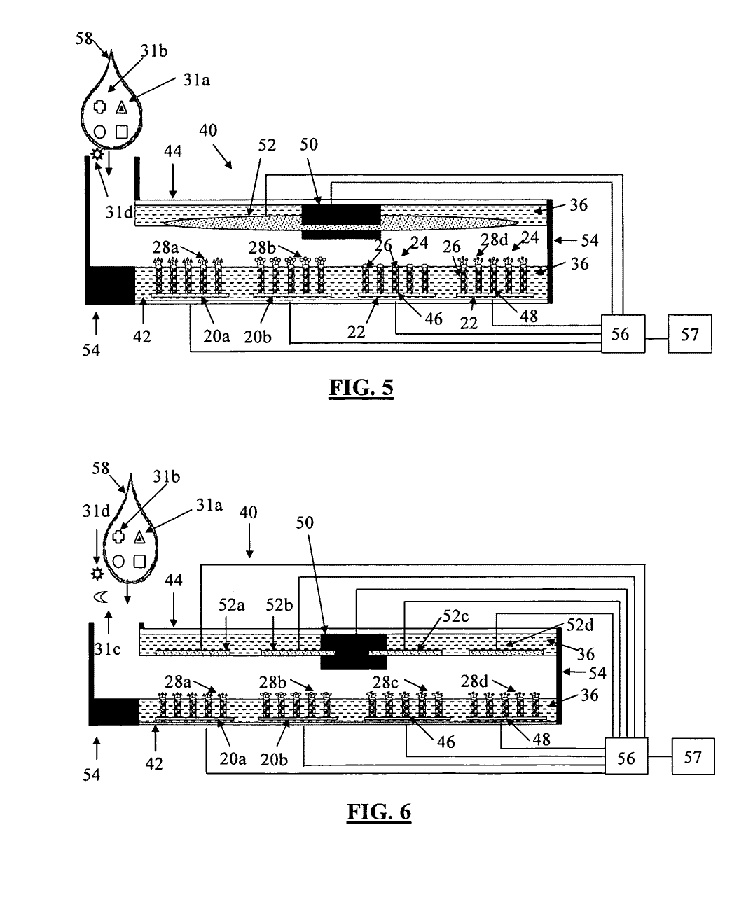

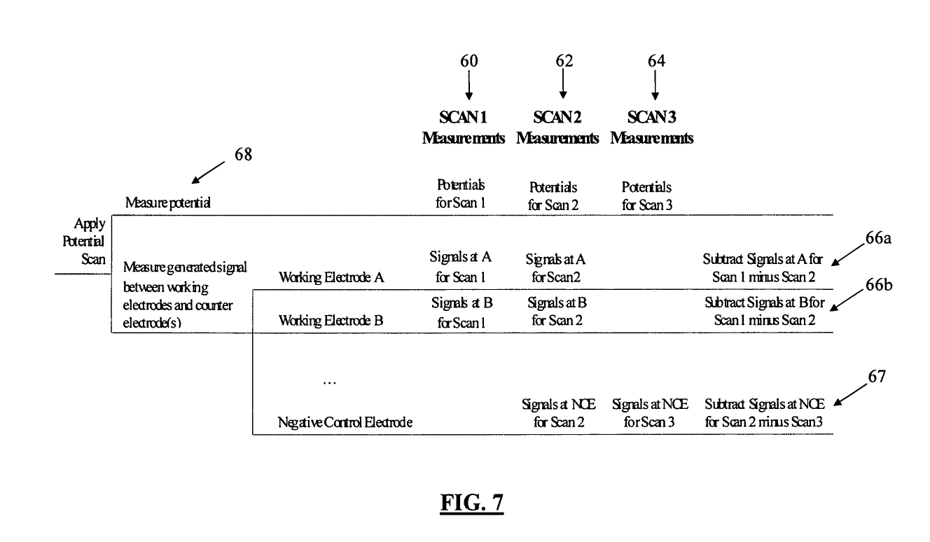

With reference to FIGS. 5 and 6, there are schematically illustrated two embodiments of an electrochemical biosensing device 40 for detecting the presence of target biomolecules in a solution, according to the present invention. The biosensing device 40 includes a bottom assembly 42 and a top assembly 44. The bottom assembly 42 has a plurality of electrodes which includes one or more working electrode 20 and at least one negative control electrode 46 for measuring background noise in the solution.

The working electrodes 20 of the bottom assembly 42 are similar to the embodiments described above or equivalents thereof. Each working electrode therefore includes an electrode pad 22, a systematic array 24 of nano-electrode wires 26 provided with biosensor probes 28. In a preferred embodiment, several working electrodes 20 are provided on a single bottom assembly 42, and at least two of these working electrodes 20 have biosensor probes 28a, 28b, etc selected to bind with different target biomolecules 31a, 31b, etc if they are present in the analyte solution 58. The biosensor probes may be oligonucleotides, nucleic acids, peptides, ligands, proteins, enzymes or any other material apt to bind with a complementary target biomolecule.

Each negative control electrode 46 preferably has a construction similar to the working electrode. The negative control electrodes therefore preferably include an electrode pad 22 defining its area and a systematic array 24 of nano-electrode wires 26 projecting vertically from the electrode pad 22. The nano-electrode wires 26 all having a same shape and size and are distributed non-randomly over the electrode pad 22, as explained above with respect to the equivalent structure on the working electrodes. In order for such an electrode to detect the background noise in the solution to provide a threshold detection limit, it may include a plurality of biosensor probes 28c (shown in FIG. 6) as explained above, attached to the top extremity of the nano-electrode wires, each biosensor probe having a bioreceptor selected to bind with one of the target biomolecules 31c intentionally added to the solution to create a binding event having weaker interaction with the target biomolecules than the biosensor probes of the working electrodes. Alternatively, the nano-electrode wires of the negative control electrodes 46 may be devoid of biosensor probes (as shown in FIG. 5), the top extremity of the nano-electrode wires being directly exposed to the solution.

In the example embodiments illustrated in FIGS. 5 and 6, the bottom assembly 42 is shown including a first working electrode 20a having biosensor probes 28a for attracting a first type of target biomolecule 31a, and a second working electrode 20b having biosensor probes 28b for attracting a second type of target biomolecule 31b different from the first type. A negative control electrode 46 is also shown. Using the example described above with 200 μm×200 μm electrode pads having around 40,000 nano-electrode wires each with a 1 μm pitch, and providing about around 100 biosensor probes per nano-electrode wires, then the working electrode 20a used to detect biomolecule A has around 4,000,000 biosensor probes for detecting biomolecule A, and the working electrode 20b used to detect biomolecule B has around 4,000,000 biosensor probes for detecting biomolecule B.

Optionally, the bottom assembly 42 of the biosensing device 40 may also include one or more positive control electrode 48 for measuring a signal from biomolecules known to be present in the solution. Each positive control electrode 48 preferably includes an electrode pad 22 having a systematic array 24 of nano-electrode wires 26 projecting vertically therefrom, and a plurality of control biosensor probes 28d each attached to the top extremity of one of the nano-electrode wires 26. Each control biosensor probe 28d includes a bioreceptor selected to bind with the control biomolecules 31d which are intentionally added to the solution to create a binding event, and an electrochemical transducer transducing this binding event into an electrical signal conducted by the corresponding nano-electrode wires 26.

It will be understood by one skilled in the art that the bottom assembly may have any appropriate number of working and control electrodes as required for a given application of the biosensing device. In its simplest form, the bottom assembly may have 2 electrodes, a working electrode for detecting the target biomolecules and a negative control electrode for measuring the background noise used to determine the detection threshold limit. In another embodiment, a biosensing device containing 16 electrodes may be considered where 14 of them are working electrodes having biosensor probes that interact with different target biomolecules, one electrode being the negative control electrode used to measure the background noise, and one electrode being the positive control electrode used to measure a positive signal from a known biomolecule intentionally added to the analyte solution to ensure that the biosensing device is functioning. For example, in such an embodiment, one working electrode can have biosensor probes used to detect the bacteria strain E. coli O157:H7, a second working electrode can have biosensor probes used to detect a specific bacteria strain of Salmonella, and 12 other working electrodes can have other biosensor probes used to detect 12 other target biomolecules. Multiple target biomolecules can therefore be detected simultaneously in parallel to provide multiplexing capability. Alternatively, sets of 2, 3 or more working electrodes on the same bottom assembly may be dedicated to a same target biomolecule for redundancy.

An insulating layer 36 extends over each electrode pad of the bottom assembly so as to surround the corresponding nano-electrode wires. Preferably, a single insulating layer 36 covers the entire bottom assembly 42, only exposing the top extremities of the nano-electrode wires 26 of each electrode.

The top assembly 44 extends over the bottom assembly 42 and includes a reference electrode 50 and at least one counter electrode 52. In the embodiment of FIG. 5, is single counter electrode 52 is provided, and is sized to extend over all of the working electrodes 20 and negative and positive control electrodes 46, 48. Alternatively, a number of counter electrodes 52a, 52b, etc corresponding to the sum total of the working electrodes 20 and control electrodes 46, 48 may be provided, with each counter electrode 52a, 52b, etc, being disposed in relative alignment with one of the working electrodes 20 or one of the control electrode 46, 48, and having similar dimensions as the corresponding working or control electrode. This embodiment is schematically illustrated in FIG. 6.

Either top assembly configuration containing one counter electrode (FIG. 5) or multiple counter electrodes (FIG. 6) can be used for either type of bottom assembly with a negative control electrode containing biosensor probes (FIG. 6) or no biosensor probes (FIG. 5).

The biosensing device 40 also includes a watertight compartment 54 housing the top and bottom assemblies 44 and 42. To use the device, an analyte solution 58 is fed through this watertight compartment 54 so as to expose the electrodes thereto.

The biosensing device 40 also includes measurement electronics 56 for applying a scan of different potentials between electrodes in the top and bottom assemblies 44 and 42 and measuring electrical signals from each working electrode 20 and each negative and positive control electrode 46 and 48. Related electronics 57 for processing these electrical signals to determine therefrom the presence of the target biomolecules and communicating the results are also provided. Each working electrode and control electrode is preferably connected through micro-circuitry and electronic connectors to the measurement electronics 56. Preferably, the measurement electronics 56 includes a potentiostat chip electrically connected to each working electrode 20, negative and positive control electrodes 46, 48, counter electrode or electrodes 52 and the reference electrode 50. The potentiostat chip may have multiple channels, each channel being dedicated to the detection of a specific type of target biomolecule. A description of a multi-channel potentiostat chip can for example be found in Zhang et al., “Electrochemical array microsystem with integrated potentiostat”, Sensors, 2005 IEEE Volume, Issue 30 October 3 November 2005.

The measurement electronics 56 are connected to the related electronics, and preferably support one or more electrochemical detection techniques. Although not illustrated in the enclosed drawing, it will be understood that the biosensing device may include any means to display the result of the detection of target biomolecules or means to transmit these results to a separate device. For example, in a portable system a display and user interface could be attached to the biosensing device or interfaced through a laptop PC, and in a wireless system a display and user interface at a distant location could be interfaced to the biosensing device through a wireless remote control module or a Supervisory Control And Data Acquisition (SCADA) system.

In one embodiment, the related electronics may further process the electrical signals to determine therefrom the concentration of the target biomolecules in the solution, as will be explained further below. In such a case, the result of the measurement taken by the device may be a concentration value for each target biomolecule, and the presence of the target biomolecule deduced from the comparison of these concentration values with a detection threshold.

In another embodiment, the related electronics may further process the electrical signals from two separate tests to determine therefrom the percentage of viable cells of the target biomolecules in the solution, as will be explained further below. In such a case, the result of the measurement taken by the device may be a % viable value for each target biomolecule, and the presence of the target biomolecule deduced from the comparison of these % viability values with a detection threshold.

As mentioned above, biosensing devices according to embodiments of the present invention may be used according to any appropriate electrochemical technique. An example of such a technique is described in more detail below.

Biosensing Method

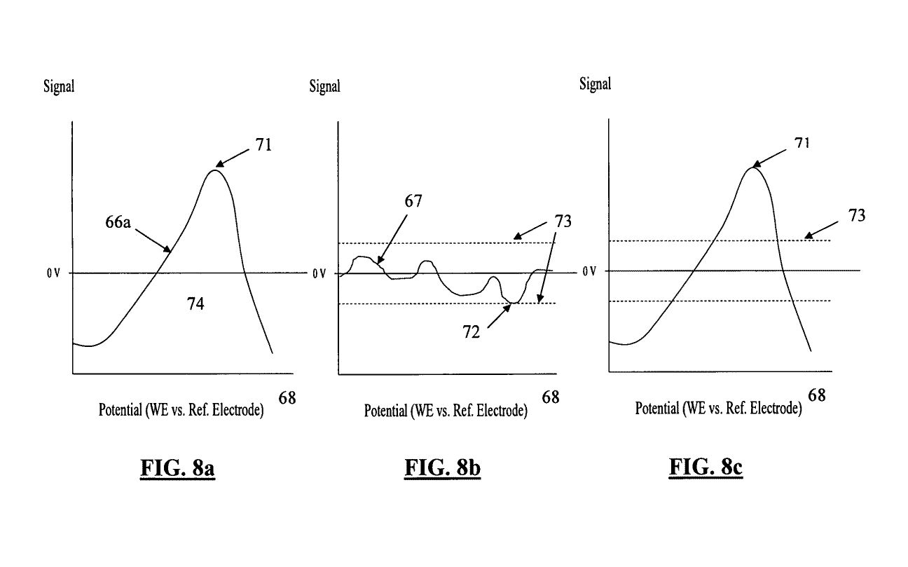

Referring to FIGS. 7, 8a, 8b and 8c, there is shown a flow chart illustrating an embodiment of a method for detecting the presence of target biomolecules in a solution, using the biosensing device as described above, according to an aspect of the present invention. In this embodiment, the bottom assembly is considered to have 4 electrodes as shown in the example of FIG. 5 or 6. With one working electrode detecting species A, one working electrode detecting species B, one negative control electrode devoid of biosensor probes, and one positive control electrode. Of course, the method described herein can be adapted to a variety of other electrode configurations, as will be readily understood by one skilled in the art.

The method first includes exposing the bottom and top assemblies of the device to the solution to be analyzed. As mentioned above, the solution is preferably pre-treated to increase the concentration of target biomolecules and to reduce the concentration of non-target biomolecules and undesired chemical molecules. Any appropriate treatment and/or microfluidics system of the like may be used to process the solution and deliver it into the watertight compartment of the biosensing device.

In one embodiment, the biosensor probes of the biosensing device are selected so that the guanine groups in the target biomolecules serve as electrochemical signal moieties that provide a small current or electrical signal produced by oxidation at around 1.02 volts versus an Ag/AgCl reference electrode. Therefore, when the potential is applied at around 1.02 volts, the current or signal is generated at the working electrode containing the biosensor probes that interact with or hybridize with target biomolecules containing a precise sequence of base pairs on the probe. This ensures high specificity of the desired target biomolecules.

In one embodiment a target biomolecule can have about 300 bases of which approximately one fourth or 75 would be inherent guanine bases. Because the number of guanine bases is very small, the generated electrical signal may be very low and difficult to accurately measure. A mediator, which also produces a signal by oxidation at around 1.02 volts, may therefore be added to the solution in order to amplify the signal from the target biomolecules. In one embodiment the mediator is the metal complex Ru(bpy)₃²⁺. The mediator can shuttle electrons from guanine bases within the hemispherical diffusion layer to ensure that all signal moieties remain active.

The method includes performing a first 60, a second 62 and a third 64 potential scan, during which the potential is applied and varied within a predetermined range in a same manner for each scan. In the preferred embodiment, for each scan a potential is applied by the measurement electronics on the working electrodes and control electrodes of the bottom assembly relative to the reference electrode on the top assembly. During each scan, the change of electrical signal such as current is measured between the each working electrode and control electrode of the bottom assembly and counter electrode(s) on the top assembly, as detailed below.

In one embodiment, AC voltammetry is used with an AC sinusoidal wave at 10 Hertz, and applied potential from 0.50 volts to 1.20 volts with a scan rate of 25 millivolts per second. This can provide data points for the generated signal before and after 1.02 volts where the peak height of the generated electrical signal is expected if target biomolecules are present in the solution.

During the first scan 60, the electric signal at each working electrode is measured across the range of potentials. Electrical signals are generated at each working electrode due to the metal ion oxidation from the mediator plus the guanine oxidation from target biomolecules if they are present in the analyte and subsequently interact with the corresponding biomolecule probes on a designated working electrode. Scan 1 therefore results in a range of signals A1 for target biomolecule A that peak at around 1.02 V and are representative of ions from the mediator and target biomolecules A binding with the biosensor probes of the first working electrode. Similarly, a range of signals B1 for target biomolecule B that peak at around 1.02 V and are representative of ions from the mediator and target biomolecules B binding with the biosensor probes of the second working electrode, is obtained.

The metal ion binding from the mediator is reversible and releases from the working electrodes after the oxidation scan. The target biomolecule binding is not reversible and does not release from the working electrodes. During the second scan 62, only the metal ions from the mediator are attracted to the working electrodes and generate a range of signals that peak at around 1.02 V. Therefore the range of signals from the target biomolecule oxidation alone 66a can be measured by subtracting the signals at each voltage point over the scan range from the first scan 60 (mediator and target biomolecule) minus the second scan 62 (mediator only). The peak signal 71 for a specific target biomolecule can be found by plotting the range of signals from the target molecules alone 66a versus the potential at the working electrode relative to the reference electrode 68.

The presence of each target biomolecule can then be determined by comparing the peak signal 71 generated from the corresponding working electrode of the target biomolecule net of the mediator as described above with a threshold detection limit 73 which is derived from the negative control electrode measuring the background noise. When the peak signal 71 from target biomolecule is greater than the threshold detection limit 73, then the target biomolecule is determined to be present in the solution.

The threshold detection limit is preferably determined by measuring the change of electrical signal from the negative control electrode obtained during the second scan 62 minus the third scan 64. During both these scans, a signal is generated at the negative control electrode containing no probes, due to the mediator metal ion oxidation. When the background noise is zero, the generated signals from both second 62 and third 64 scans should be zero. In almost all cases the background noise is greater than zero, and can be obtained by finding the greatest difference 72 from each voltage point over the scan range when subtracting the signal from the second scan (mediator only) minus the third scan (mediator only) 67. This value is extrapolated over the entire scan range 73 and used as the threshold detection limit to detect the presence of the biomolecules from the peak signals 71 or other measures.

In one embodiment the threshold detection limit is set equal to the peak background noise. Alternatively, fluctuations of the background noise over different potentials can be accounted for and the threshold detection limit set to three times the standard deviation of the background noise signal amplitude weighted average.

Optionally, the method above may include determining the concentration of each target biomolecule detected in the solution from a comparison of the measured changes of electrical signal from the corresponding working electrodes with predetermined values from samples of known concentrations of these target biomolecules.

A useful value for determining the concentration of a target biomolecule from the electrical signal at a given working electrode may be calculated using different approaches. FIGS. 8a, 8b, and 8c show the signal 66a value at a given working electrode, compensated for mediator binding and background noise, as a function of the potential between the corresponding working electrode and the reference electrode. In one approach, the measured electrical signal value is set to the peak signal 71 of the generated curve. Alternatively, the area 74 under the curve 66a may be used. Other approaches could also be devised without departing from the scope of the present invention.

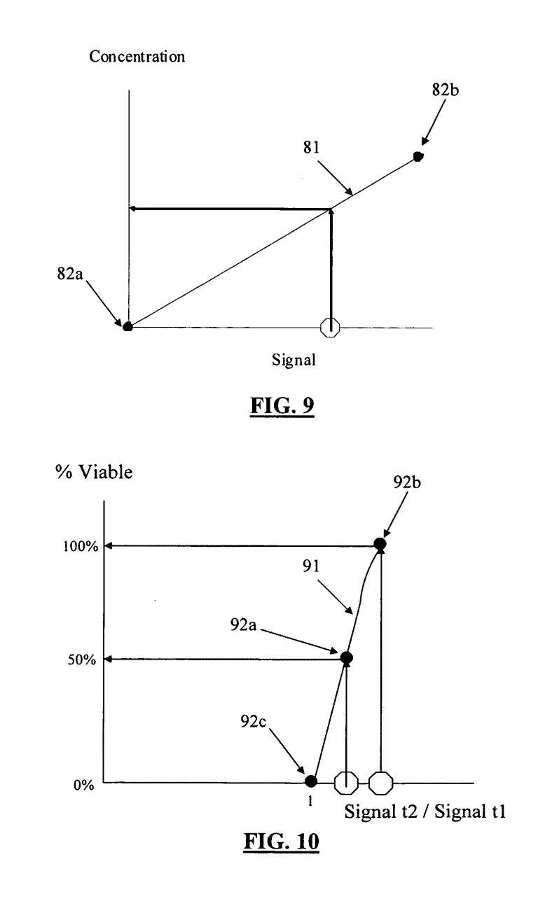

In one embodiment, a linear relationship 81 is established between peak signals 71 and biomolecule concentrations as shown in FIG. 9. At least two standard predetermined signal values 82a, 82b, . . . could be used to obtain such a relationship. For example, the concentration of a given type of target biomolecules may be set equal to the peak signal from the corresponding working electrode multiplied by the ratio of the concentration of Standard 1 minus the concentration of Standard 2, divided by the peak signal of Standard 1 minus the peak signal of Standard 2. In another embodiment, the peak signal for each parameter may be replaced with the area 74 generated under the curve 66a. In another embodiment, a nonlinear relationship can exist between peak signals and biomolecule concentrations. In this embodiment a suitable formula would be determined for extrapolating peak signals into concentration levels.

Of course, the method above could be adapted for use with a different number and configuration of electrodes on the bottom assembly. For example, electrical signals from more than one working electrode could be obtained and combined to increase detection of a given type of target biomolecules. Electrical signals from one or more positive working electrode could also be factored in the calculating of the background or threshold limit.

Optionally, the method above may include determining the percentage of cells of each target biomolecule in the solution that are capable of dividing and increasing in number (% viable). This may be important to some applications since the presence and concentration of a specific toxin producing strain of microorganism species that is infective to humans may be in the solution, and knowing that a percentage of its cells are viable and able to reproduce will indicate the risk of infectivity from the sample.

The percentage of viable cells is preferably determined from a comparison of the measured changes of electrical signal in the test solution compared with predetermined values from samples for known % viable cells of these target biomolecules. In one embodiment the initial solution is separated into a first and a second portion, defining equivalent samples. The first portion is immediately prepared and tested for the presence and concentration of target biomolecules using the preparation processes and detection method described above. The second portion is subjected to conditions stimulating cell division for a period of time. This may for example involve adding nutrients, for example one or more sugar such as glucose or the like, or exposing the second portion to a favorable environment such as heat. The conditions to which the second portion is exposed are preferably selected to relieve stressed microorganisms and encourage cell division. The period of time for which the second portion is set aside preferably corresponds to approximately the time needed to double the amount of target microorganisms, or a partial amount, for example 10 to 30 minutes. The second portion is then prepared and tested for the presence and concentration of target biomolecules using the preparation processes and detection method described above with a second unused biosensing device. If the biosensing device includes working electrodes dedicated to the detection of different target biomolecules, the method above for detecting the % viable cells could be used for each type of biomolecules.

A useful value for determining the % viable cells of a target biomolecule is the magnitude of the electrical signal generated in the detection method. In one approach, the % viable cells of a target biomolecule can be determined from the ratio of the peak electrical signal from the second portion, divided by the peak electrical signal of the first portion for the same target biomolecule. This ratio reflects a growth in the number of cells if the ratio is greater than one whereby a portion of the cells are viable; or a decline in the number of cells if the ratio is less than one and there are virtually no viable cells. Alternatively, the ratio of the area under this curve may be used.

Other approaches could also be devised without departing from the scope of the present invention. This can include measuring the change in the ratio of signals after non-viable are separated out of the second sample, or by using a messenger RNA probe to measure the amount and/or change of messenger RNA.

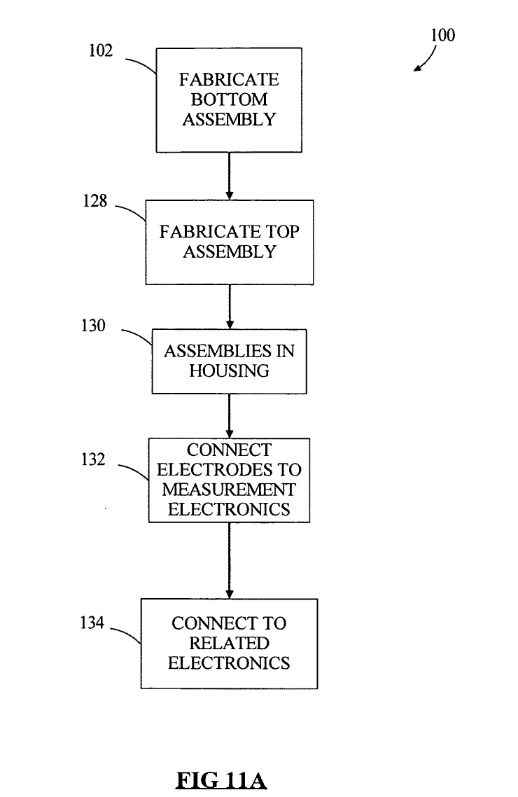

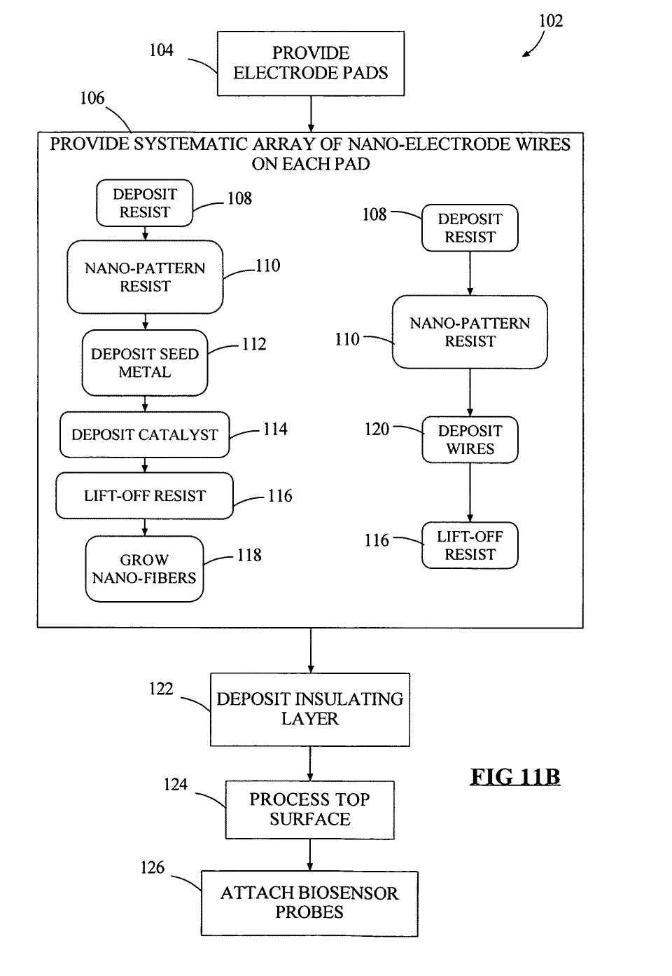

In one embodiment, a near linear relationship 91 is established between the ratio of peak signals and % viable cells as shown in FIG. 10. At least two standard predetermined signal values could be used to obtain such a relationship, including the value where 100% of the cells are viable 92b and where 50% of the cells are viable 91a. For example, the % viable cells of a given type of target biomolecules may be set equal to the peak signal ratio from the corresponding working electrode multiplied by the ratio of the ratio of Standard 1 minus the ratio of Standard 2, divided by the peak signal of Standard 1 minus the peak signal of Standard 2. In another embodiment, the peak signal for each parameter may be replaced with the area 74 generated under the curve 66a. In another embodiment, a nonlinear relationship can exist between peak signals and % viable ratios. In this embodiment a suitable formula would be determined for extrapolating peak signals into % viable levels.

Fabrication Method

In accordance with another aspect of the present invention, there is also provided a method for the fabrication of one or multiple working electrodes on a bottom assembly of a biosensing device.

It will be understood by one skilled in the art that it is usually advantageous to fabricate all the electrodes of one or more bottom assemblies simultaneously, and the embodiment of the method described below will be explained with reference to the fabrication of several working and reference electrodes on one bottom assembly. However, any fabrication process in which at least one working electrode is fabricated according to the method of the present invention is considered within its scope.

It will also be understood by one skilled in the art that it is usually advantageous to fabricate many bottom assemblies and/or top assemblies simultaneously as individual dies on a semiconductor wafer. However the embodiment of the method described below will be explained with reference to the fabrication of a single top assembly and a single bottom assembly, but any fabrication process in which one or many bottom assemblies or top assemblies is fabricated according to the method of the present invention is considered within its scope.

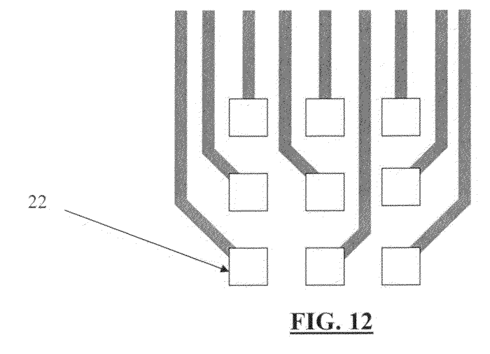

Referring to FIG. 11A there is shown a high level flow chart illustrating an embodiment of a method 100 for fabricating a biosensing device as described above, according to an aspect of the present invention.

The method first includes fabricating a bottom assembly 102 for such a device. A preferred embodiment of such a step is shown in FIG. 11B. The method for fabricating the bottom assembly 102 first includes providing a plurality of electrode pads 104 on a substrate, such as a silicon wafer. In one embodiment, the surface of the substrate may be previously insulated, for example with a low-pressure chemical vapor deposition (LPCVD) of either a silicon nitride or silicon oxide layer of about 500 nm thickness. Other insulating materials can also be used. Each electrode pad defines the area of the corresponding electrode, whether a working electrode or a control electrode. The electrode pad is preferably made of a conductive or semi-conductive material such as chromium, platinum, titanium or related conductive metals.

In one embodiment of the invention, the fabrication of the electrode pads may include depositing, for example by spin-coating, a resist layer over the substrate. The resist layer is then patterned to form a plurality of cavities. Each cavity defines the area of one of the electrode pads. Micro-patterning techniques known in the art such as photolithography may be used to create micrometer sized square, rectangular, circular or otherwised-shaped patterns for the electrode pads and for corresponding micro-circuitry. The circuitry preferably connects each electrode pad to a metal connector or pin for electrical measurements. A conductive material is then deposited over the resist layer and within the cavities. In one embodiment, a metal film of about 200 nm thickness is deposited using E-beam evaporation. The method may then include lifting-off the resist layer and conductive material thereon. This leaves only the material deposited in the cavities on the substrate, which forms the electrode pads and circuitry.

FIG. 12 is a photograph of bottom assembly at this stage of fabrication. On the substrate are 9 micro-sized electrode pads in a 3×3 array. Each micro-electrode pad 22 is connected with micro-circuitry to electrical connectors or pins.

The method next includes providing a systematic array of nano-electrode wires 106 on each electrode pad. As explained above, the nano-electrode wires project vertically from the corresponding electrode pad. Within a given systematic array, all the nano-electrode wires have a same shape and size and are distributed non-randomly over the corresponding electrode pad.

In accordance with one embodiment of the invention, the fabrication of the systematic arrays of nano-electrode wires includes depositing a resist layer 108 over the substrate, preferably a thermopolymer through spin coating, and nano-patterning the resist layer 110 over each electrode pad to form vertically indented nanocavities. The nanocavities are given a size, shape and distribution corresponding to the desired predetermined size, shape and distribution of the nano-electrode wires. For example, the cavity size may selected from the same value between 50 and 100 nm, the cavity shape may be cylindrical, and the spacing is selected from the same value between 1 and 5 μm.

Nanopatterning is preferably performed using Nanolmprint Lithography Hot Embossing. A negative of each nanocavity to be patterned on the bottom assembly's working electrodes and control electrodes is fabricated onto a master stamp. The stamp patterns the resist under a predetermined temperature and pressure to form the desired nanocavities. Other nanopatterning techniques can also be used such as E-beam lithography or photolithography.

The method then preferably includes depositing a seed metal 112 over the entire resist layer and in the nanocavities. The seed layer is preferably embodied by a metal film, for example of about 10 to 50 nm in thickness deposited using any appropriate technique such as for example E-beam evaporation. In one embodiment chromium is selected as the metal of the seed layer. A catalyst material is then deposited 114 over the seed metal. E-beam evaporation could also be used for this second deposition, and the catalyst material may for example be a layer of about 10 to 100 nm thickness. The catalyst material is selected to promote the growth of carbon nanofibers between the seed and catalyst layers as will be further explained below. In one example nickel is used as the catalyst material, although various other types of metal could be used depending on the material and structure to be used for the nano-electrodes.

Following the steps above, the seed metal and catalyst material in the nanocavities of each electrode pad will defined a systematic array of nano-dots. The method then includes lifting-off the resist layer 116 from the substrate and electrode pads, therefore leaving only the nano-dots on the electrode pads. Multi-walled carbon nanofibers are then grown 118 between the seed metal and catalyst material of each nano-dot, for example using plasma-enhanced chemical vapor deposition (PECVD).

Unlike carbon nanotubes, carbon nanofibers form a series of closed graphitic shells along the fiber axis similar to a bamboo-like structure that seals the inner channel and prevents any liquid from entering. It is known in the art that the height of the carbon nanofibers grown using PECVD is proportional to the diameter of the catalyst. In one embodiment carbon nanofibers are grown to approximately 3.2 μm in height using a 40× aspect ratio and the same 80 nanometer diameter and circular shape for virtually every nano-dot produced from nanocavities patterned with Nanolmprint Lithography. In this embodiment since the carbon nanofibers are cylindrical, upright, perpendicular to the substrate, and at least 1 μm apart from neighboring carbon nanofibers due to the electric field effect, they do not contact neighboring carbon nanofibers or enter the neighboring hemispherical diffusion layer which reduces the sensitivity of the signal as in randomly distributed forests of carbon nanofibers. The actual yields will be in line with fabricated products in the semiconductor industry.

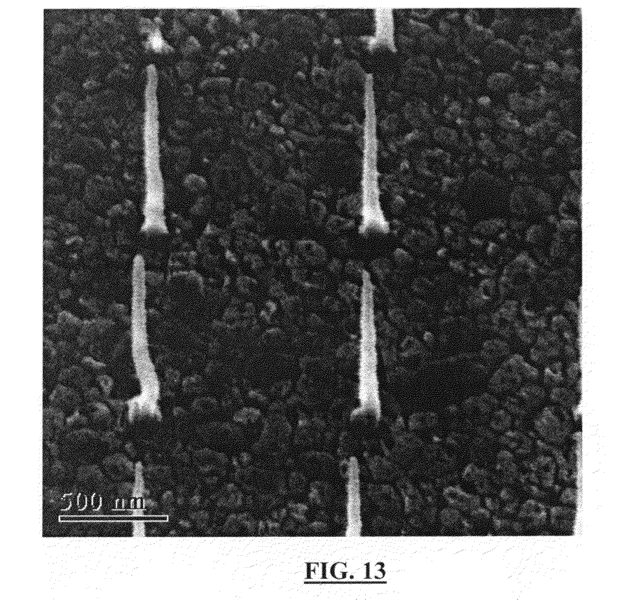

FIG. 13 is a photograph of an electrode pad 22 containing a systematic array of multiwalled carbon nanofibers 26 which are used as nano-electrodes wires.

Other embodiments can grow nano-electrode wires from other materials such as silicon, zinc oxide, tin oxide, indium oxide and related materials, and fabricating different shapes such nanotubes, nanocones and nanowhiskers.

In an alternative embodiment, the nano-electrode wires may be deposited 120 directly in the nanocavities formed from the nanopatterning of the resist layer. In this case, the vertically indented nanocavities have an elongated shape, corresponding to the desired elongated shape of the nano-electrode wires, and the systematic array of nano-electrode wires of each electrode pad is obtained by depositing a conductive or semi-conductive material over the resist layer and into the nanocavities. The material in the nanocavities can then directly define the nano-electrode wires. The resist layer is lifted-off 116 from the electrode pads, leaving the nano-electrode wires thereon. The conductive or semi-conductive material may be copper, aluminum, indium, antimony or related materials.

Once the nano-electrode wires have been fabricated, the fabrication method includes depositing an insulating layer 122 over each electrode pad, so as to surround the nano-electrode wires thereon. In the preferred embodiment the insulating layer extends over the entire substrate including the portion of the substrate containing the electrode pads. The insulating layer is preferably embodied by a dielectric SiO₂ film deposited using thermal chemical vapor deposition of tetra-ethylorthosilicate (TEOS CVD) to at least the height of the nano-electrode wires. The insulating layer therefore fills in the space between the nano-electrode wires and encapsulates each wire. Preferably, the insulating layer also covers the remaining substrate surface to prevent the non-specific adsorption of target biomolecules in the solution to the side surfaces of the nano-electrode wires, which can render false positive readings.

For each of the electrode pads to become a working electrode, the method then includes processing the top surface 124 of the working electrode, at this point constituted of the electrode pad provided with a systematic array of nano-electrode wires surrounded by the insulating layer, to prepare the top extremities of the nano-electrode wires to receive and subsequently attach biosensor probes. This processing may include several steps. In one embodiment, this processing first includes planarizing the top surface of the insulating layer and the top extremities of the nano-electrode wires, for example using chemical mechanical planarization (CMP) and polishing over the entire top surface. This first step removes the excess insulating layer and the ceilings formed on the nano-electrode wires by the catalyst material resulting in a roughly even top surface. The processing of the top surface of the electrode may then include removing portions of the top extremities of said nano-electrode wires, for example through reactive ion etching (RIE), which exposes the tips of the nano-electrode wires.

The method then includes attaching a plurality of biosensor probes 126 to the top extremities of said nano-electrode wires. As explained above, each biosensor probe includes a bioreceptor selected to bind with a complementary target biomolecule to create a binding event, and an electrochemical transducer transducing this binding event into an electrical signal conducted by the corresponding nano-electrode wire.

There can be a substantial adsorption of non-target biomolecules on the top surface of the insulating layer. Chemical treatment can eliminate the adsorption of non-target biomolecules and reduce false positive results but can also increase false negative results because of its insulating capabilities. To reduce the chance of false negatives, the top surface of the insulating layer can be treated by chemically applying layers of passivated protective moieties to the top surface of each working electrode, the protective moieties being selected to prevent an adsorption of non-specific biomolecules. For example, bovine serum albumin (BSA) or poly ethylene glycol (PEG) can be used. The top extremities of the nano-electrode wires are then preferably etched to remove the passivated protective moieties. This may for example be performed by exposing the nano-electrode wires to nitric acid followed by sodium hydroxide, while applying a voltage of about 1.5 Volts to the nano-electrode wires. This allows —COOH groups to form on the tips of the nano-electrode wires. The working electrodes are then exposed to a solution containing the biosensor probes with coupling agents. For example, a mixture of Amine linked oligonucleotide probes with coupling agents may be used to form covalent bonds where electroactive guanine bases are substituted by nonelectroactive inosine bases in the biosensor probes. Different solutions containing different biosensor probes and coupling agents may be spotted on different working electrodes to obtain a multiplexed biosensing device detecting several target biomolecules simultaneously. A selective spotter may be used to expose each working electrode individually with an appropriate solution containing the desired biosensor probes. The spotting of probes would be repeated for each working electrode and control electrode for spotting the desired probes and coupling agents.

Referring back to FIG. 11A, the top assembly of the device is fabricated 128 either concurrently, before or after the fabrication of the bottom assembly 102. The method for fabricating the top assembly 128 first includes providing a plurality of electrode pads on a substrate, such as a silicon wafer. In one embodiment, the surface of the substrate may be previously insulated, for example with a low-pressure chemical vapor deposition (LPCVD) of either a silicon nitride or silicon oxide layer of about 500 nm thickness. Other insulating materials can also be used. Each electrode pad defines the area of the corresponding electrode, whether one reference electrode and one or more counter electrodes. The electrode pad is preferably made of a conductive or semi-conductive material such as platinum, titanium, chromium or related conductive metals.

In one embodiment of the invention, the fabrication of the electrode pads may include depositing, for example by spin-coating, a resist layer over the substrate. The resist layer is then patterned to form a plurality of cavities. Each cavity defines the area of one of the electrode pads. Micro-patterning techniques known in the art such as photolithography may be used to create micrometer sized square, rectangular, circular or otherwised-shaped patterns for the electrode pads and for corresponding micro-circuitry. The circuitry preferably connects each electrode pad to a metal connector or pin for electrical measurements. A conductive material is then deposited over the resist layer and within the cavities. In one embodiment, a metal film, preferably platinum of about 200 nm thickness is deposited using E-beam evaporation. The method may then include lifting-off the resist layer and conductive material thereon. This leaves only the material deposited in the cavities on the substrate, which forms the electrode pads and circuitry.

The reference electrode is preferably further processed with a screen print or other deposition technique to apply a layer of silver/silver chloride solution on the platinum electrode. A mask may be used to prevent the solution from depositing on the other areas of the top assembly.

Once done the top and bottom assemblies are joined within a waterproof housing 130, preferably a polymer. The electrodes of both assemblies are connected to measurement electronics 132, themselves connected to related electronics 134, as defined above.

Of course, numerous variations could be made to the embodiments described above without departing from the scope of the invention, as defined in the appended claims.

1. A working electrode on a bottom assembly of a biosensing device, the working electrode comprising:
an electrode pad defining an area of the working electrode;
a systematic array of nano-electrode wires projecting vertically from said electrode pad, said nano-electrode wires all having a same shape and size and being distributed non-randomly over said electrode pad;
a plurality of biosensor probes each attached to an extremity of one of said nano-electrode wires opposite said electrode pad, each biosensor probe comprising a bioreceptor selected to bind with a complementary target biomolecule to create a binding event, and an electrochemical transducer transducing said binding event into an electrical signal conducted by the corresponding nano-electrode wire; and
an insulating layer extending over said electrode pad so as to surround said nano-electrode wires while exposing said biosensor probes.

2. The working electrode according to claim 1, wherein the electrode pad is made of a conductive material.

3. The working electrode according to claim 1, wherein the electrode pad has a length and a width each of about 0.5 to 2000 μm.

4. The working electrode according to claim 1, wherein the nano-electrode wires are made of carbon, silicon, zinc oxide, tin oxide indium oxide, copper, aluminum, indium, or antimony.

5. The working electrode according to claim 1, wherein the nano-electrode wires are shaped as nanofibers, nanotubes, nanocones, or nanowhiskers.

6. The working electrode according to claim 1, wherein the nano-electrode wires are carbon nanofibers having a multi-walled structure.

7. The working electrode according to claim 1, wherein the nano-electrode wires each have a circular cross-section and a diameter of about 50 to 150 nm.

8. The working electrode according to claim 1, wherein the nano-electrode wires each have a height of about 0.1 to 20 μm.

9. The working electrode according to claim 1, wherein the nano-electrode wires each have a height to diameter aspect ratio between 20 and 60.

10. The working electrode according to claim 1, wherein said nano-electrode wires have a same distance therebetween.

11. The working electrode according to claim 10, wherein said same distance is selected between about 1 μm and 5 μm.

12. The working electrode according to claim 1, wherein said biosensor probe is an oligonucleotide, nucleic acid, peptide, ligand, protein or enzyme.

13. The working electrode according to claim 1, wherein said biosensor probe is a 16S ribosomal RNA oligonucleotide or biomolecule.

14. The working electrode according to claim 1, wherein the insulating layer is made of SiOₓ, Si_{y}N_{z}, epoxy, wax or parylene, where x, y and z are positive numbers.

15. A method for the fabrication of a working electrode for a biosensing device on a substrate, the method comprising:
a) providing an electrode pad on a portion of the substrate, thereby defining an area of the working electrode;
b) providing a systematic array of nano-electrode wires projecting vertically from said electrode pad, said nano-electrode wires all having a same shape and size and being distributed non-randomly over said electrode pad;
c) depositing an insulating layer over said electrode pad and surrounding said nano-electrode wires;
d) processing a top surface of said working electrode to prepare top extremities of said nano-electrode wires to receive biosensor probes; and
e) attaching a plurality of biosensor probes to said top extremities of said nano-electrode wires, each biosensor probe comprising a bioreceptor selected to bind with a complementary target biomolecule to create a binding event, and an electrochemical transducer transducing said binding event into an electrical signal conducted by the corresponding nano-electrode wire.

16. The method according to claim 15, wherein the providing a systematic array of nano-electrode wires of b) comprises:
i. depositing a resist layer over said substrate and electrode pad; and
ii. nano-patterning said resist layer over the electrode pad to form vertically indented nanocavities having a size, shape and distribution corresponding to the predetermined size, shape and distribution of the nano-electrode wires.

17. The method according to claim 16, wherein the nano-patterning of b)ii is performed using NanoImprint Lithography-Hot Embossing.

18. The method according to claim 16, wherein the providing a systematic array of nano-electrode wires of b) further comprises, after the nano-patterning of ii:
iii. depositing a seed metal over said resist layer and in the nanocavities;
iv. depositing a catalyst material over said seed metal, the seed metal and catalyst material in the nanocavities defining a systematic array of nano-dots;
v. lifting-off the resist layer from the substrate and electrode pad, leaving the nano-dots on the electrode pad; and
vi. growing multi-walled carbon nanofibers between the seed metal and catalyst material of each nano-dots.

19. The method according to claim 18, wherein the growing multi-walled carbon nanofibers of b)vi comprises using plasma-enhanced chemical vapor deposition.

20. The method according to claim 16, wherein the vertically indented nanocavities have an elongated shape, and wherein the providing a systematic array of nano-electrode wires of b) further comprises, after the nano-patterning of ii:
iii. depositing a conductive or semiconductive material over said resist layer and into said nanocavities, the metal in said nanocavities defining the nano-electrode wires; and
iv. lifting-off the resist layer from the electrode pad, leaving the nano-electrode wires thereon.

21. The method according to claim 15, wherein the insulating layer is a dielectric SiO₂ film, the depositing of c) being performed using thermal chemical vapor deposition of tetra-ethylorthosilicate.

22. The method according to claim 15, wherein the processing a top surface of said working electrode of d) comprises planarizing a top surface of the insulating layer and top extremities of the nano-electrode wires.

23. The method according to claim 22, wherein said planarizing performed using chemical mechanical planarization and polishing.

24. The method according to claim 18, wherein the processing a top surface of said working electrode of d) comprises:
i. removing an excess of the insulating layer from the surface of said working electrode; and
ii. removing the catalyst material from top extremities of said nano-electrode wires.

25. The method according to claim 22, wherein the processing a top surface of said working electrode of d) comprises, after said planarizing, removing portions of the top extremities of said nano-electrode wires.

26. The method according to claim 25, wherein said removing portions of the top extremities of said nano-electrode wires is performed using reactive ion etching.

27. The method according to claim 15, wherein the attaching a plurality of biosensor probes to said top extremities of said nano-electrode wires of e) comprises:
i. chemically applying layers of passivated protective moieties to the top surface of the working electrode, said protective moieties being selected to prevent an adsorption of non-specific biomolecules;
ii. electrochemically etching the top extremities of said nano-electrode wires to remove said passivated protective moieties therefrom;
iii. exposing said working electrode to a solution containing said biosensor probes and coupling agents, the biosensor probes attaching to said top extremities of said nano-electrode wires.

28. The method according to claim 27, wherein the passivated protective moieties comprise poly ethylene glycol or bovine serum albumin.

29. The method according to claim 27, wherein the electrochemically etching of e)ii. comprises treating said nano-electrode wires with nitric acid followed by sodium hydroxide, while applying a voltage of about 1.5 Volts to said nano-electrode wires.

30. An electrochemical biosensing device for detecting a presence of target biomolecules in a solution, said biosensing device comprising:
a bottom assembly comprising at least one negative control electrode for measuring background noise in said solution and at least one working electrode, each working electrode comprising:
an electrode pad defining an area of the working electrode;
a systematic array of nano-electrode wires projecting vertically from said electrode pad, said nano-electrode wires all having a same shape and size and being distributed non-randomly over said electrode pad;
a plurality of biosensor probes each attached to an extremity of one of said nano-electrode wires opposite said electrode pad, each biosensor probe comprising a bioreceptor selected to bind with one of said target biomolecules to create a binding event, and an electrochemical transducer transducing said binding event into an electrical signal conducted by the corresponding nano-electrode wire; and
an insulating layer extending over said electrode pad so as to surround said nano-electrode wires while exposing said biosensor probes;
a top assembly extending over said bottom assembly and comprising a reference electrode and at least one counter electrode;
a watertight compartment housing said top and bottom assemblies;
measurement electronics for applying a scan of different potentials between electrodes in said top and bottom assemblies and measuring electrical signals from each working electrode and each negative control electrode; and
related electronics for processing said electrical signals to determine therefrom the presence of said target biomolecules.

31. The biosensing device according to claim 30, wherein the bottom assembly comprises a plurality of said working electrodes, the biosensors of at least two of said working electrodes being selected to bind with different types of said target biomolecules.

32. The biosensing device according to claim 30, wherein the electrode pad of each of said working electrodes is made of a conductive material.

33. The biosensing device according to claim 30, wherein the electrode pad of each of said working electrodes has a length and a width each of about 0.5 to 2000 μm.

34. The biosensing device according to claim 30, wherein the nano-electrode wires of each of said working electrodes are made of carbon, silicon, zinc oxide, tin oxide indium oxide, copper, aluminum, indium, or antimony.

35. The biosensing device according to claim 30, wherein the nano-electrode wires of each of said working electrodes are shaped as nanofibers, nanotubes, nanocones, or nanowhiskers.

36. The biosensing device according to claim 30, wherein the nano-electrode wires of each of said working electrodes are carbon nanofibers having a multi-walled structure.

37. The biosensing device according to claim 30, wherein the nano-electrode wires each of said working electrodes have a circular cross-section and a diameter of about 50 to 150 nm.

38. The biosensing device according to claim 30, wherein the nano-electrode wires of each of said working electrodes each have a height of about 0.1 to 20 μm.

39. The biosensing device according to claim 30, wherein the nano-electrode wires of each of said working electrodes each have a height to diameter aspect ratio between 20 and 60.

40. The biosensing device according to claim 30, wherein said nano-electrode wires of each of said working electrodes have a same distance therebetween.

41. The biosensing device according to claim 40, wherein said same distance is selected between about 1 μm and 5 μm.

42. The biosensing device according to claim 30, wherein said biosensor probe is an oligonucleotide, nucleic acid, peptide, ligand, protein or enzyme.

43. The biosensing device according to claim 30, wherein said biosensor probe is a 16S ribosomal RNA oligonucleotide or biomolecule.

44. The biosensing device according to claim 30, wherein the insulating layer of each of said working electrodes is made of SiOₓ, Si_{y}N_{z}, epoxy, wax or parylene, where x, y and z are positive numbers.

45. The biosensing device according to claim 30, wherein each of said negative control electrodes comprises:
an electrode pad defining an area of the negative control electrode;
a systematic array of nano-electrode wires projecting vertically from said electrode pad, said nano-electrode wires all having a same shape and size and being distributed non-randomly over said electrode pad, each of said nano-electrode wire being devoid of biosensor probes; and
an insulating layer extending over said electrode pad so as to surround said nano-electrode wires while exposing top extremities thereof.

46. The biosensing device according to claim 30, wherein each of said negative control electrodes comprises:
an electrode pad defining an area of the negative control electrode;
a systematic array of nano-electrode wires projecting vertically from said electrode pad, said nano-electrode wires all having a same shape and size and being distributed non-randomly over said electrode pad,
a plurality of biosensor probes each attached to an extremity of one of said nano-electrode wires opposite said electrode pad, each biosensor probe comprising a bioreceptor selected to bind with one of said target biomolecules to create a binding event having weaker interaction with the target biomolecules than the biosensor probes of the working electrodes, and an electrochemical transducer transducing said binding event into an electrical signal conducted by the corresponding nano-electrode wire; and
an insulating layer extending over said electrode pad so as to surround said nano-electrode wires while exposing said biosensor probes.

47. The biosensing device according to claim 30, wherein said bottom assembly further comprises at least one positive control electrode for measuring a signal from biomolecules known to be present in said solution.

48. The biosensing device according to claim 47, wherein each positive control electrode comprises:
an electrode pad defining an area of the positive control electrode;
a systematic array of nano-electrode wires projecting vertically from said electrode pad, said nano-electrode wires all having a same shape and size and being distributed non-randomly over said electrode pad;
a plurality of control biosensor probes each attached to an extremity of one of said nano-electrode wires opposite said electrode pad, each control biosensor probe comprising a bioreceptor selected to bind with said control biomolecules to create a binding event, and an electrochemical transducer transducing said binding event into an electrical signal conducted by the corresponding nano-electrode wire; and
an insulating layer extending over said electrode pad so as to surround said nano-electrode wires while exposing said control biosensor probes.

49. The biosensing device according to claim 30, comprising a number of said counter electrodes corresponding to a sum total of said working electrodes and IS said control electrodes, each of said counter electrode being disposed in relative alignment with one of said working electrodes or one of said control electrodes, and having similar dimensions as the corresponding working or control electrode.

50. The biosensing device according to claim 30, comprising a single one of said counter electrodes, sized to extend over all of said working electrodes and control electrodes.

51. The biosensing device according to claim 30, wherein the related electronics further process said electrical signals to determine therefrom a concentration of said target biomolecules.

52. The biosensor device according to claim 30, wherein the measurement electronics comprise a potentiostat chip electrically connected to each of said working electrodes, control electrodes, counter electrodes, and reference electrode.

53. The biosensor device according to claim 52, wherein the potentiostat chip comprises multiple channels, each of said channels being dedicated to the detection of a specific type of target biomolecules.

54. A method for the fabrication of a bottom assembly of an electrochemical biosensing device for detecting a presence of target biomolecules in a solution, the method comprising:
a) providing a plurality of electrode pads on a substrate, said electrode pads defining at least one negative control electrode for measuring background noise in said solution and at least one working electrode;
b) providing a systematic array of nano-electrode wires on each of said electrode pads, said nano-electrode wires projecting vertically from the corresponding electrode pad, said nano-electrode wires of one of said systematic arrays all having a same shape and size and being distributed non-randomly over the corresponding electrode pad;
c) depositing an insulating layer over each of said electrode pad surrounding the nano-electrode wires thereon;
for each of said working electrodes:
d) processing a top surface of said working electrode to prepare top extremities of said nano-electrode wires to receive biosensor probes; and
e) attaching a plurality of biosensor probes to said top extremities of said nano-electrode wires, each biosensor probe comprising a bioreceptor selected to bind with a complementary target biomolecule to create a binding event, and an electrochemical transducer transducing said binding event into an electrical signal conducted by the corresponding nano-electrode wire.

55. The method according to claim 54, wherein the providing a plurality of electrode pads of a) comprises:
i. depositing a resist layer over said substrate;
ii. patterning said resist layer to form a plurality of cavities, each cavity defining an area of one of said electrode pads;
iii. depositing a conductive material over said resist layer and within said cavities;
iv. lifting-off the resist layer and conductive material thereon, leaving the conductive material deposited within said cavities on said substrate.

56. The method according to claim 54, wherein the providing a systematic array of nano-electrode wires on each electrode pad of b) comprises:
i. depositing a resist layer over said substrate; and
ii. nano-patterning said resist layer over each of the electrode pads to form vertically indented nanocavities having a size, shape and distribution corresponding to the predetermined size, shape and distribution of the nano-electrode wires.

57. The method according to claim 56, wherein the nano-patterning of b)ii is performed using NanoImprint Lithography-Hot Embossing.

58. The method according to claim 56, wherein the providing a systematic array of nano-electrode wires on each electrode pad of b) further comprises, after the nano-patterning of ii:
iii. depositing a seed metal over said resist layer and in the nanocavities;
iv. depositing a catalyst material over said seed metal, the seed metal and catalyst material in the nanocavities of each electrode pad defining a systematic array of nano-dots;
v. lifting-off the resist layer from the substrate and electrode pads, leaving the nano-dots on said electrode pads; and
vi. growing multi-walled carbon nanofibers between the seed metal and catalyst material of each nano-dot.

59. The method according to claim 58, wherein the growing multi-walled carbon nanofibers of b)vi comprises using plasma-enhanced chemical vapor deposition.

60. The method according to claim 56, wherein the vertically indented nanocavities have an elongated shape, and wherein the providing a systematic array of nano-electrode wires of each electrode pad of b) further comprises, after the nano-patterning of ii:
iii. depositing a conductive or semi-conductive material over said resist layer and into said nanocavities, the material in said nanocavities defining the nano-electrode wires; and
iv. lifting-off the resist layer from the electrode pads, leaving the nano-electrode wires thereon.

61. The method according to claim 54, wherein the insulating layer is a dielectric SiO₂ film, the depositing of c) being performed using thermal chemical vapor deposition of tetra-ethylorthosilicate.

62. The method according to claim 54, wherein the processing a top surface of each working electrode of d) comprises planarizing a top surface of the insulating layer and top extremities of the nano-electrode wires.

63. The method according to claim 62, wherein said planarizing performed using chemical mechanical planarization and polishing.

64. The method according to claim 58, wherein the processing a top surface of each working electrode of d) comprises:
i. removing an excess of the insulating layer from the surface of said working electrodes; and
ii. removing the catalyst material from top extremities of said nano-electrode wires.

65. The method according to claim 62, wherein the processing a top surface of each working electrode of d) comprises, after said planarizing, removing portions of the top extremities of said nano-electrode wires.

66. The method according to claim 65, wherein said removing portions of the top extremities of said nano-electrode wires is performed using reactive ion etching.

67. The method according to claim 54, wherein the attaching a plurality of biosensor probes to said top extremities of said nano-electrode wires of e) comprises:
i. chemically applying layers of passivated protective moieties to the top surface of each working electrode, said protective moieties being selected to prevent an adsorption of non-specific biomolecules;
ii. electrochemically etching the top extremities of said nano-electrode wires to remove said passivated protective moieties therefrom;
iii. exposing said working electrodes to a solution containing said biosensor probes and coupling agents.

68. The method according to claim 67, wherein the passivated protective moieties comprise poly ethylene glycol or bovine serum albumin.

69. The method according to claim 67, wherein the electrochemically etching of e)ii. comprises treating said nano-electrode wires with nitric acid followed by sodium hydroxide, while applying a voltage of about 1.5 Volts to said nano-electrode wires.

70. The method according to claim 67, wherein e iii. comprises using a selective spotter to expose each working electrode to different solutions containing different biosensor probes and coupling agent.

71. A method for the fabrication of an electrochemical biosensing device for detecting a presence of target biomolecules in a solution, the method comprising:
a) fabricating a bottom assembly according to the method of claim 54;
b) fabricating a top assembly, including providing a plurality of electrode pads on a substrate, said electrode pads defining at least one counter electrode, and one reference electrode;
c) joining the bottom assembly and top assembly within a water-proof housing;
d) connecting the electrodes of the bottom and top assemblies to measurement electronics for applying a scan of different potentials between electrodes in said top and bottom assemblies and measuring electrical signals from each working electrode and each negative control electrode; and
e) connecting the measurement electronics to related electronics for processing said electrical signals to determine therefrom the presence of said target biomolecules.

72. A method for detecting a presence of target biomolecules in a solution using the biosensing device of claim 30, comprising:
a) exposing said bottom and top assemblies to said solution;
b) performing a first, a second and a third detection scan, whereby the potential difference between the electrodes of the top and bottom assembly is varied within a predetermined range in a same manner for each of said detection scans;
c) measuring a change of electrical signal from each of said working electrodes for the first and second detection scans;
d) measuring a change of electrical signal from each of said negative control electrodes for the second and third detection scans;
e) determining said presence of the target biomolecules from said changes of electrical signal measured at c) and d).

73. The method according to claim 72, further comprising:
f) determining a concentration of said target biomolecules in said solution that are present in e) from a comparison of the changes of electrical signal measured at c) and predetermined values for known concentrations of said target biomolecules.

74. The method according to claim 72, further comprising determining a % viable cells of said target biomolecules in said solution, said determining comprising:
performing a preliminary step of dividing said solution into a first and a second portion;
performing steps a) through f) on the first portion of said solution immediately thereafter;
subjecting the second portion to conditions stimulating cell division for a period of time;
performing steps a) through f) on said second portion after said period of time; and
comparing ratio of electrical signals from the second and first portions to ratios of electrical signals from predetermined values for known % viable values of said target biomolecules.

75. The method according to claim 74, wherein the subjecting the second portion to conditions stimulating cell division comprises at least one of adding nutrients to said second portion and exposing said second portion to a favorable environment.

76. The method according to claim 75, wherein said nutrients comprise at least one sugar and said favorable environment comprises heat.

77. The method according to claim 72, wherein the predetermined range of the potential scan between the electrodes of the top and bottom assemblies is around about 1.02 volts.

78. The method according to claim 72, wherein the bottom assembly of said biosensing device comprises a plurality of said working electrodes, the bioreceptors on the biosensors of at least two of said working electrodes being selected to bind with different types of said target biomolecules, the determining of e) of said method comprising the determining the presence of said target biomolecules of each of said type.

79. The method according to claim 78, further comprising:
f) determining, when present in said solution, a concentration of said target biomolecules of each of said type from a comparison of the changes of electrical signal measured at c) for the corresponding working electrodes, and predetermined values for known concentrations of said target biomolecules.

80. The method according to claim 78, further comprising determining a % viable cells of said target biomolecules of each of said type in said solution, said determining comprising:
performing a preliminary step of dividing said solution into a first and a second portion;
performing steps a) through 0 on the first portion of said solution immediately thereafter using working electrodes corresponding to each of said types of target biomolecules;
subjecting the second portion to conditions stimulating cell division for a period of time;
performing steps a) through f) on said second portion after said period of time using other working electrodes corresponding to each of said types of target biomolecules; and
comparing, for each type of target biomolecules, ratio of corresponding electrical signals from the second and first portions to ratios of electrical signals from predetermined values for known % viable values of said target biomolecules of the corresponding type.

81. The method according to claim 80, wherein the subjecting the second portion to conditions stimulating cell division comprises at least one of adding nutrients to said second portion and exposing said second portion to a favorable environment.

82. The method according to claim 81, wherein said nutrients comprise at least one sugar and said favorable environment comprises heat.
